(19) United States
(12) Patent Application Publication
(10) Pub. No.: US 2024/0424173 A1
(43) Pub. Date: Dec. 26, 2024

(54) SURGICAL DEVICES USING MULTIPLE MEMORY SHAPE MEMORY MATERIALS

(71) Applicant: SMARTER ALLOYS INC., Cambridge (CA)

(72) Inventors: Mohammad Ibrahem KHAN, Cambridge (CA); Michael Lawrence KUNTZ, Cambridge (CA); Alanna KIRCHNER, Cambridge (CA)

(21) Appl. No.: 18/563,951

(22) PCT Filed: May 30, 2022
(86) PCT No.: PCT/CA2022/050866

§ 371 Date: Nov. 24, 2023

Related U.S. Application Data
(60) Provisional application No. 63/194,488, filed on May 28, 2021.

(51) Int. Cl. A61L 31/14; A61L 29/02; A61L 29/14; A61L 31/02; A61M 25/01; A61M 25/09

(52) U.S. Cl. CPC A61L 31/14; A61L 29/02; A61L 29/14; A61L 31/022; A61L 2400/16; A61M 25/0158; A61M 2025/09158

(57) ABSTRACT

Surgical devices that are made from a shape memory alloy (SMA) that is then processed via a multiple memory material process to impart at least altered property via at least one processed region. In use, when a predetermined temperature is applied to the surgical device, the at least one processed regions responds to the predetermined temperature and provides a predetermined functionality.

### CROSS-REFERENCE TO OTHER APPLICATIONS

The present disclosure claims priority from U.S. Provisional Application No. 63/194,488 filed May 28, 2021 which is hereby incorporated by reference.

### FIELD

The present disclosure relates generally to shape memory materials, and more specifically to surgical devices using shape memory materials.

### BACKGROUND

Shape memory materials include a variety of materials that move between states when subject to temperature changes. Two of the key properties of shape memory materials are the shape memory effect (SME) and pseudoelasticity (PE). The most common shape memory materials are metal alloys but other shape memory materials are also known. The SME often describes the ability of the material to “remember” its original shape upon heating from a martensitic state to above an austenitic finish temperature (temperature induced phase transformation). PE describes the non-linear recoverable deformation up to 8% strain beyond its linear elastic region (stress induced phase transformation). Nitinol (NiTi), a shape memory alloy made of nearly equiatomic parts nickel and titanium, is commonly used in medical devices because, in addition to these two properties, NiTi exhibits excellent biocompatibility due to the titanium oxide layer that forms on its surface.

Surgical devices such as guidewires and catheters have benefited from the incorporation of NiTi. Many guidewires available on the market use NiTi as the core material or as a sleeve on the tip providing the benefit of improved kink resistance and an improved level of flexibility to navigate anatomies compared to commonly used stainless steel guidewires. The compromise, however, can be inferior torqueability and crossability. Catheter manufacturers have incorporated NiTi to reinforce an outer jacket, however these devices face similar trade-offs. For these reasons, surgeons may be required to use multiple devices in one procedure to achieve the desired treatment thereby increasing operating time.

There are also various other surgical devices used for manipulation in surgical interventions that have incorporated NiTi. For example, tissue retractors are used to visualize intrathoracic structures and provide an entry point for instrumentation during cardiac surgery. The challenge with tissue retractors is avoiding tissue trauma while effectively securing the opening and having the device return to shape following deformation during insertion. Heart stabilizers are used to perform off-pump surgery and prevent or reduce the need for a heart-lung machine, stabilizing the heart to allow it to beat during open-heart surgery. The challenge for stabilizers is achieving a steady bloodless area comparable to a heart that is arrested with cardioplegia. Clamps are used for many surgical procedures, designed to temporarily occlude veins or arteries. They must be accurately positioned and provide occlusive forces while avoiding vessel damage from excessive clamping forces. NiTi valve resection blades have been developed to cut away a damaged heart valve and prepare the area so that a prosthetic valve can be placed and secured without resulting in leakage or valve drift. The challenge is minimizing resection time, avoiding injury to surrounding tissues and sizing correctly. Retrieval devices are used to non-invasively remove foreign objects in the body, including in vessels, and must be able to manipulate to grasp the object and effectively compress the target object into a catheter or secure it for safe removal. Embolic filters are used to capture and retrieve debris to prevent embolisms, and must conform to the vessel geometry, have an adequate pore size to allow for blood flow and recover debris without its release.

NiTi is also used for various implantable devices. One of the most widely accepted uses of NiTi in the medical field is in stents. NiTi stents offer the advantage of being able to self-expand without the use of a balloon and have superior performance in terms of resistance to buckling and radial/bending compliance. However, NiTi stents pose a challenge in accurate placement as they are typically loaded when deployed from a catheter, often causing a spring-forward that can result in inaccurate placement. NiTi percutaneous valves have been developed to replace diseased heart valves with the goal of avoiding blood thinners (required for typical mechanical valves) or additional surgeries (required for bioprosthetic valves as they will experience calcification) but run the challenge of achieving a sufficient cycle life. Annuloplasty bands have incorporated NiTi cores, with a design challenge being the balance between flexibility for non-invasive deployment and stiffness to provide strength of the valve.

For all surgical or implantable devices, the general challenge is to try and reduce operating times, decrease the invasiveness of a given procedure, reduce the risk of complications and reduce the need for additional treatments. As such, there is an on-going need for improved surgical devices.

Therefore, there is provided novel surgical devices using shape memory materials that overcomes disadvantages of current solutions.

### SUMMARY

The disclosure is directed at surgical devices with improved navigation and functionality, and, in particular to various devices used in surgery formed from shape memory materials that has been multiple memory material processed and to methods of forming/making these surgical devices.

According to an aspect herein there is provided a surgical device including two or more shape memory processed sections having differing SME and/or PE properties and methods of forming/making the surgical device. In some embodiments, the surgical devices according to the disclosure herein can be used for surgical interventions that may overcome at least some of the existing challenges with navigation, anatomical compatibility and complexity or length of time for a procedure.

According to another aspect of the disclosure, there is provided a surgical device including at least one processed region having altered properties to provide a predetermined functionality; wherein the surgical device is made from a shape memory alloy (SMA); and wherein the SMA is processed via a multiple memory material process to impart the at least one processed region into the SMA.

In another aspect, the surgical device further includes at least two processed regions. In yet another aspect, one of the at least two processed regions provides a functionality at a first predetermined temperature and another of the at least two processed regions provides a functionality at a second predetermined temperature. In another aspect, the first predetermined temperature and the second predetermined temperature are different. In a further aspect, the first predetermined temperature and the second predetermined temperature are the same.

In another aspect of the disclosure, the surgical device is one of a guidewire, a catheter, an adjustable diameter ring, a tissue retractor, an annuloplasty band, a heart stabilizer, a clamp, a frame for an embolic filter, a stent, a valve, a clip or a drug delivery device.

In a further aspect, when the surgical device is a catheter, the at least one processed region is near a tip of the catheter whereby when a predetermined temperature is applied to the catheter, the tip of the catheter bends at varying angles. In yet a further aspect, when the surgical device is a catheter, the at least one processed region includes pleats or folds whereby when a predetermined temperature is applied to the catheter, a diameter of the catheter increases to a target diameter. In another aspect, when the surgical device is a guidewire, the at least one processed region includes at least two processed regions located on a diameter of the guidewire. In yet another aspect, the at least two processed regions are opposite each other.

In another aspect, when the surgical device is an adjustable diameter ring, in response to a predetermined temperature, the at least one processed region bends to increase a diameter of the adjustable diameter ring. In yet another aspect, when the surgical device is a clamp, in response to a predetermined temperature, the at least one processed region bends from an open position to a closed position. In a further aspect, when the surgical device is a frame for an embolic filter, in response to a predetermined temperature, the at least one processed region bends from an open position to a closed position. In yet another aspect, when the surgical device is a stent, the at least one processed region includes multiple processed regions that respond to different predetermined temperatures. In another aspect, when one of the different predetermined temperatures is applied to the stent, a portion of the multiple processed regions expands in response to the application of the different predetermined temperature.

In another aspect, when the surgical device is a drug delivery device that includes a set of micro-pores filled in a pressure condition, in response to a predetermined temperature, the at least one processed region exposes the micro-pores to release a drug stored in the set of micro-pores. In a further aspect, when the surgical device is a drug delivery device that includes a set of micro-pores filled under a vacuum condition, in response to a predetermined temperature, the at least one processed region exposes the micro-pores to create a suction to draw matter into the micro-pores.

In yet a further aspect, the SMA is a form of a sheet, a wire or a tube. In yet another aspect, the surgical device is post-processed after the SMA is processed via the multiple memory material process. In another aspect, post processing includes cold work, heat treatment, tumbling, thermal cycling, training or electropolishing.

Other aspects and features will become apparent, to those ordinarily skilled in the art, upon review of the following description of some exemplary embodiments.

### BRIEF DESCRIPTION OF THE DRAWINGS

Embodiments of the present disclosure will now be described, by way of example only, with reference to the embedded Figures.

FIGS. 1a and 1b are schematic diagrams of multiple memory material (MMM) processes;

FIG. 2a is a schematic diagram of a conventional pigtail catheter;

FIG. 2b is a schematic diagram steerable pigtail catheter;

FIG. 3 is a cross-section of a MMM processed guidewire;

FIG. 4 is a cross-section of a MMM processed catheter core;

FIG. 5 is a series of schematics of a compressible self-expanding catheter;

FIGS. 6a to 6c are schematic diagrams of a surgical device with processed curve regions;

FIG. 7a is a schematic diagram of an expandable tip with regions processed in a martensite phase;

FIG. 7b is a schematic diagram of the expandable tip of FIG. 7a with regions processed in an austenite phase;

FIG. 8a is a schematic diagram of an adjustable ring;

FIG. 8b is a schematic diagram of the adjustable ring of FIG. 8a with regions processed in an austenite phase;

FIG. 9a is a schematic diagram of a heart stabilizer in an open position;

FIG. 9b is a schematic diagram of the heart stabilizer of FIG. 9a in a closed position;

FIG. 10a is a schematic side view of a frame of an embolic filter in an open position;

FIG. 10b is a schematic side view of the frame of FIG. 10a in a closed position;

FIGS. 11a to 11d are schematic diagrams of a stent at various temperature stages;

FIGS. 12a to 12e are schematic diagrams of a NiTi butterfly valve;

FIG. 13a is a schematic diagram of a clip in an initial position;

FIG. 13b is a schematic diagram of the clip of FIG. 13a in a first memory position;

FIG. 13c is a schematic diagram of the clip of FIG. 13a in a second memory position;

FIGS. 14a and 14b are schematic diagrams of a MMM processed SMA wire with micro-pores covered with valves; and

FIGS. 15a to 15f are schematic cross-sections of a MMM processed SMA with drug eluting capabilities.

### DETAILED DESCRIPTION OF THE EMBODIMENTS

The following description with reference to the accompanying drawings is provided to assist in understanding of example embodiments as defined by the claims and their equivalents. The following description includes various specific details to assist in that understanding but these are to be regarded as merely examples. Accordingly, those of ordinary skill in the art will recognize that various changes and modifications of the embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used to enable a clear and consistent understanding. Accordingly, it should be apparent to those skilled in the art that the following description of embodiments is provided for illustration purpose only and not for the purpose of limiting the disclosure as defined by the appended claims and their equivalents.

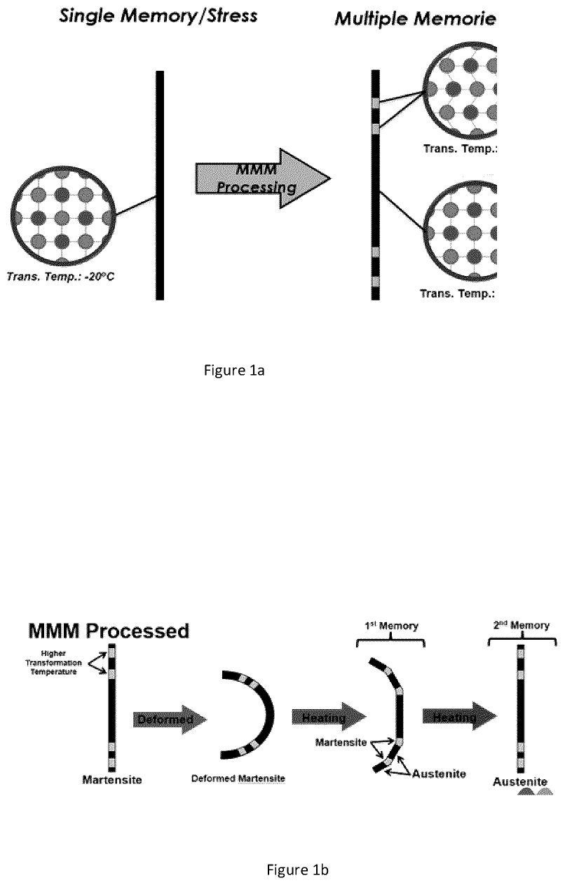

The disclosure is directed at surgical devices that are made from a shape memory alloy (SMA) material that is then processed to impart at least one different transformation temperatures to processed regions of the SMA. One example of multiple memory material (MMM) processing is disclosed in U.S. Pat. No. 9,186,853, granted Nov. 17, 2015, which is hereby incorporated by reference. Schematic diagrams of the MMM process are shown in FIGS. 1a and 1b.

Processing the SMA with MMM technology allows for precisely tuning the local transformation temperatures of processed regions of SMAs. In another embodiment, MMM processing alters the local properties of different locations or areas of the SMA to be different than its original properties. In one embodiment, this allows multiple transformation temperatures/properties to be utilized for SMAs, allowing for a dynamic response at distinct locations. Specifically, MMM technology is a method for applying energy, such as, but not limited to laser or heat processing, to a local area of a shape memory material to adjust the local structure and chemistry. Another example of MMM processing may include the addition of alloying elements during the laser process. This provides one or more additional transformation temperatures and modified pseudo-elastic properties of the treated local area or processed region. It can also enable additional functionality, such as local radio opacity through the addition of Pt during processing. The remaining unaffected material still exhibits its original functional properties. Hence, additional memories, or transformation temperatures, can be embedded into a monolithic SMA component, which in turn enables additional functionality. This makes it possible to fabricate a monolithic SMA that can operate passively or actively in a wide range of temperatures. The SMAs may also be MMM processed with three-dimensional (3D) printing methods.

SMAs have unique properties with two being the shape memory effect (SME) and pseudoelasticity (PE). The SME results from the ability of an alloy to transform from a rigid, high temperature austenite phase to a malleable, low temperature martensite phase during cooling. Once a high temperature shape is trained, or shape set, into an SMA component or material, in the austenite phase, it can further be cooled to its martensite phase and deformed. When the material is cooled below a martensitic finish temperature (Mf), it is entirely martensite and easily deformed. Upon heating above an austenitic finish temperature (Af), the material becomes entirely austenite and returns to its trained shape, exhibiting large forces. In some cases, there may be alternate phases, such as R-phase, that are observed in the material which may replace the martensitic phase in the transformation.

The present disclosure relates to various types of surgical devices and methods for forming the same. Generally speaking, when referring to heating of the device, this may be achieved actively (i.e., via an electric current, heated fluid, inductive heating, etc.), passively (by exposure to body temperature), or a combination of both. In the current disclosure, MMM SMA may be used for any of various types of surgical and/or medical devices included in the embodiments shown, as well as others not disclosed. Also, while the disclosure specifically discusses the use of a SMA, such as, but not limited to, NiTi, CuNiTi, or others, similar principles can be applied to other shape memory materials.

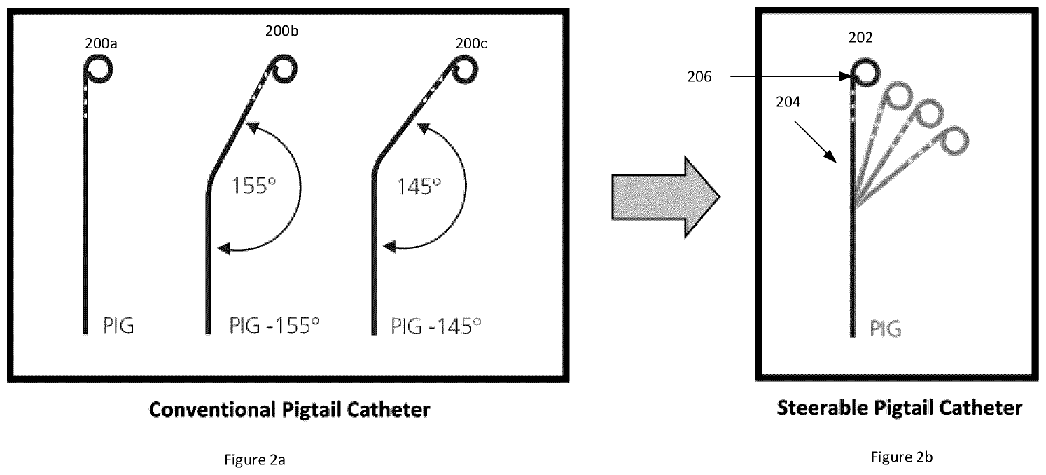

In one embodiment, the disclosure is directed at the use of MMM processed SMA for creating or manufacturing guidewires and/or catheters. In the following description, use of the word catheter may include guidewires as well. FIGS. 2a and 2b provide examples of a conventional pigtail catheter (FIG. 2a) and a novel steerable pigtail catheter (FIG. 2b) in accordance with the disclosure.

As shown in FIG. 2a, conventional pigtail catheters 200 are manufactured with only a single angle which cannot be changed or manipulated once the catheter is finished. Known pigtail catheters include a straight PIG 200a, a PIG—155° 200b and a PIG—145° 200c.

Unlike conventional pigtail catheters, one embodiment of a catheter in accordance with the disclosure may be seen as a steerable pig catheter 202. The steerable pig catheter 202 is made from a SMA that has been MMM processed to impart multiple transformation temperatures (such as in the form of processed regions) to the catheter. In other embodiments, the MMM processing may impart altered local properties at the processed regions that provide predetermined functionality in response to certain conditions. During the MMM processing, a region 204 near a tip 206 of the catheter 202 is processed to impart a further transformation temperature at the region 204 to create a steerable tip that can be bent at varying angles when the catheter is exposed to a predetermined temperature. The catheter 202 may be bent either in the same axes or multiple axes to create three-dimensional (3D) motion. In this manner, the steerable pig catheter 202 can be manufactured to include any angle required which provides more flexibility in comparison with conventional pig catheters. After subjecting the SMA portion or portions of the catheter through the MMM process, there may be a need for post processing to finish the medical device. Examples of post processing may include, but are not limited to, cold work, heat treatment, tumbling, thermal cycling, training and/or electropolishing.

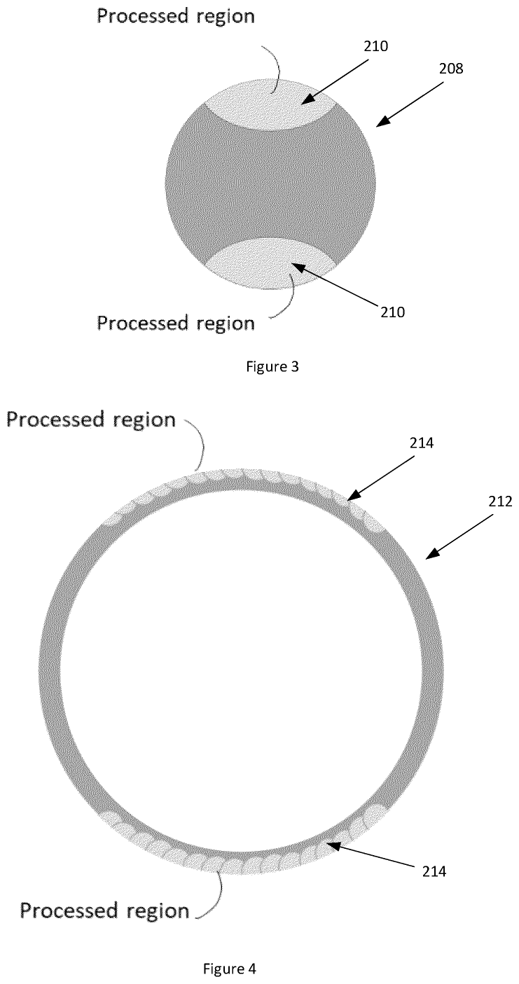

In other embodiments, such as schematically shown in FIG. 3 (which is a cross-section of a guidewire), the guidewire 208 may be MMM processed along opposing locations 210 on the guidewire such that rotating the guide wire by approximately 90° allows it to move through a path either with more flexibility or more strength. The unprocessed regions (i.e. length of device) retain its original properties.

In another embodiment, such as schematically shown in FIG. 4 (which is a cross-section of a catheter 212), the catheter 212 can be processed via MMM at different locations 214. It is understood that while the different locations 210 (FIG. 3) and 214 (FIG. 4) are opposed to each other, they may be at any place on the diameter of the medical device and not necessarily opposite each other.

In a further embodiment of a catheter, FIG. 5 shows a set of cross-sections of a compressible, self-expanding catheter 216 that is manufactured from an SMA that has been MMM processed. In one embodiment, the MMM processing may be to pleat and fold the SMA catheter 216 upon itself radially to decrease an overall diameter prior to deployment of the catheter which may prevent or reduce the need for a guidewire.

After the catheter is inserted into a patient's body, the catheter may be heated and in response to the applied temperature or applied predetermined temperature, the catheter 216 may then expand to a target diameter to allow for delivery/drainage via the catheter 216. The catheter 216 may also be laser processed to reduce stress in locations or regions of higher strain. In other embodiments, the catheter may expand in response to different conditions, not necessarily temperature based.

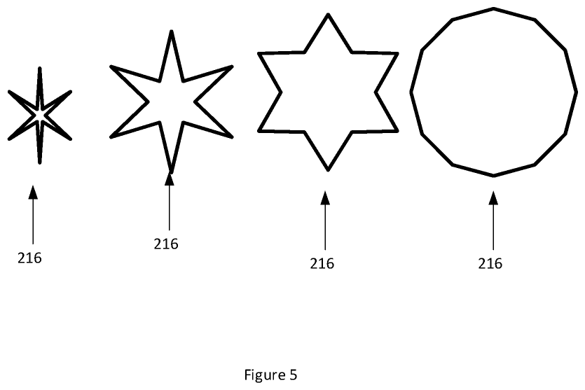

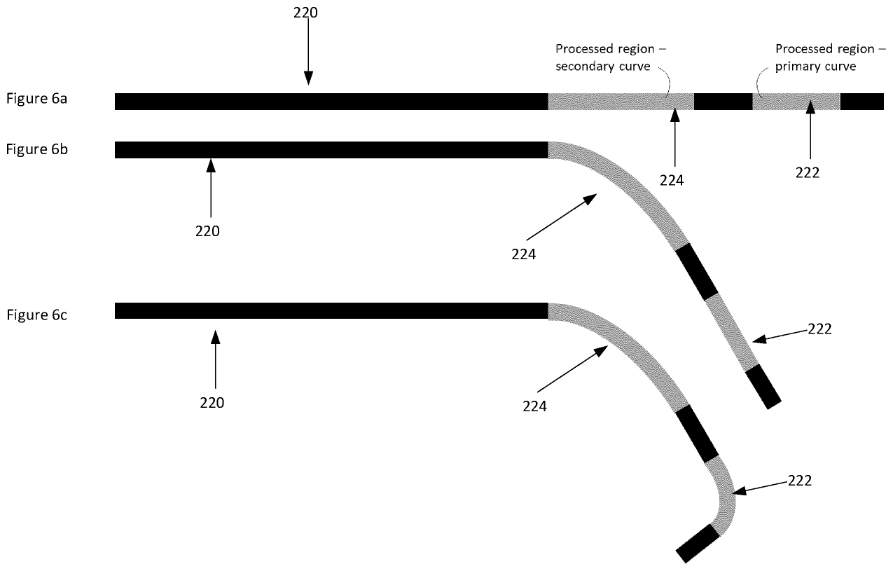

In another embodiment of a medical device that is manufactured from a MMM processed SMA, FIGS. 6a to 6c show a catheter that has two differently processed regions. As shown in FIG. 6a, a catheter 220 (made of an SMA) is MMM processed to include a first processed region 222 representing a primary curve section and a second processed region 224 representing a secondary curve section. In FIG. 6a, the processed regions are shown in a martensite phase at an initial temperature. As a higher temperature is applied to the catheter, as schematically shown in FIG. 6b, the second processed region 224 curves in response to the higher temperature or a first predetermined temperature or temperature range. It is understood that in this example, the second processed region 224 is in an austenite phase at a temperature higher than the initial temperature while the first processed region 222 does not react at this higher temperature. The response by the second processed region 224 allows the catheter to perform a first function at this higher temperature. As the applied temperature continues to increase, as schematically shown in FIG. 6c, the first processed region 222 curves in response to the increasing temperature or a second predetermined temperature or temperature range. In other words, FIG. 6c shows the first 222 and second 224 processed regions in an austenite phase at a temperature than is higher than the one applied to the catheter in FIG. 6b. This enables the catheter to perform a second function in response to this increasing temperature. As will be understood, if there are further processed regions, they may provide other functionality or functionalities to the catheter when predetermined temperatures are applied to the catheter. In some examples, multiple processed regions may curve or react at the same temperature and each processed region does not necessarily have to react to its own specific temperature.

In another embodiment, different regions of a catheter can be MMM processed for primary, secondary, ternary (or more) curves or shapes that can deform and return to their remembered shape at varying predetermined temperatures or temperature ranges (i.e. return to each of the curves sequentially as temperature rises). Alternatively, the processed regions may all have the same transformation temperatures.

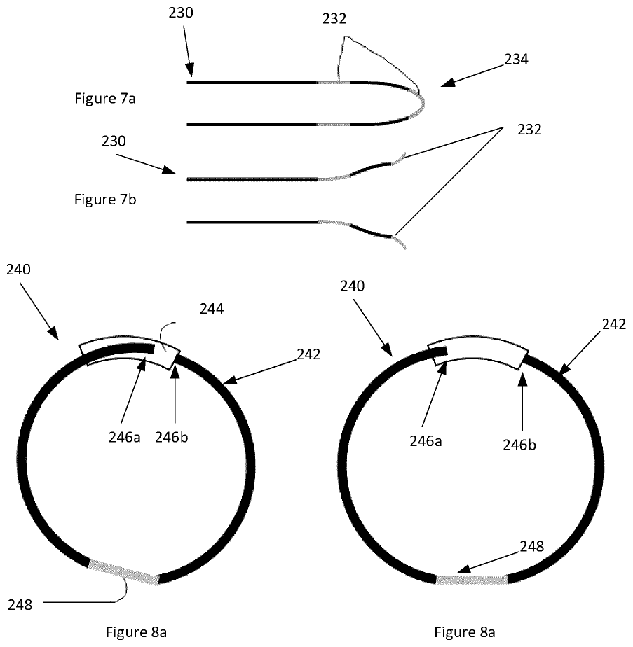

Turning to FIGS. 7a and 7b, schematic diagrams of an expandable catheter tip in a martensite phase (FIG. 7a) and an austenite phase (FIG. 7b) are provided. In use, the tip of the catheter may expand to secure to vessel walls during stent deployment. As will be understood, the catheter 230 of FIGS. 7a and 7b includes processed regions 232 that have been processed via a MMM process such that the catheter tip 234 responds to an applied predetermined temperature enabling the tip 234 to expand during deployment. Initially, the catheter tip 234 is compact during deployment (FIG. 7a) and then opens up (FIG. 7b), when heated or exposed to a temperature higher than the temperature applied in FIG. 7a (such as a predetermined temperature), to apply force to vessel walls, for example, to secure the catheter in place, to be used, for example, to support deployment of self-expanding stent and prevent or reduce the likelihood or spring-forward caused by stent exiting cannula.

Turning to FIGS. 8a and 8b, other types of medical devices that may be made out of a SMA and then MMM processed in accordance with the disclosure are tissue retractors and/or annuloplasty bands or an adjustable diameter ring for tissue retractors and/or annuloplasty bands. These types of devices are typically a ring that is not a continuous circle such that there is a break in the ring covered by a SMA material (such as a NiTi sleeve) that is rigidly attached to one of the ends of the opening. FIG. 8a shows an adjustable diameter ring for a tissue retractor, annuloplasty band and the like with an initial diameter at an initial temperature and FIG. 8b shows the adjustable diameter ring in response to a different temperature (such as the predetermined temperature) with the processed section in an austenite phase.

As shown in FIGS. 8a and 8b, the device 240 is a “broken” ring 242 of SMA material that includes a SMA sleeve 244 that is connected to two ends 246a and 246b of the ring 242. As discussed above, the sleeve 244 is rigidly connected to one end 246a of the two ends 246. The ring 242 includes at least one processed region 248 where the ring has been MMM processed. When an increasing temperature (or the predetermined temperature) is applied to the ring 242, the processed region 248 actuates in response to the applied increasing temperature and starts to bend, pulling the unsecured end 246b further away from the secured end 246a but remains within the sleeve 244 thereby increasing a diameter of the opening, or ring 242 which provides an adjustable diameter feature to the medical device. This provides an advantage over current devices which may not have the dynamic, or on-the-fly adjustability. In an alternative embodiment, the medical device may not include a sleeve whereby the two ends simply move away from each other in response to the applied predetermined temperature. In yet another embodiment, the shape of the ring 242 may not be a complete circle and can be a portion of a circle.

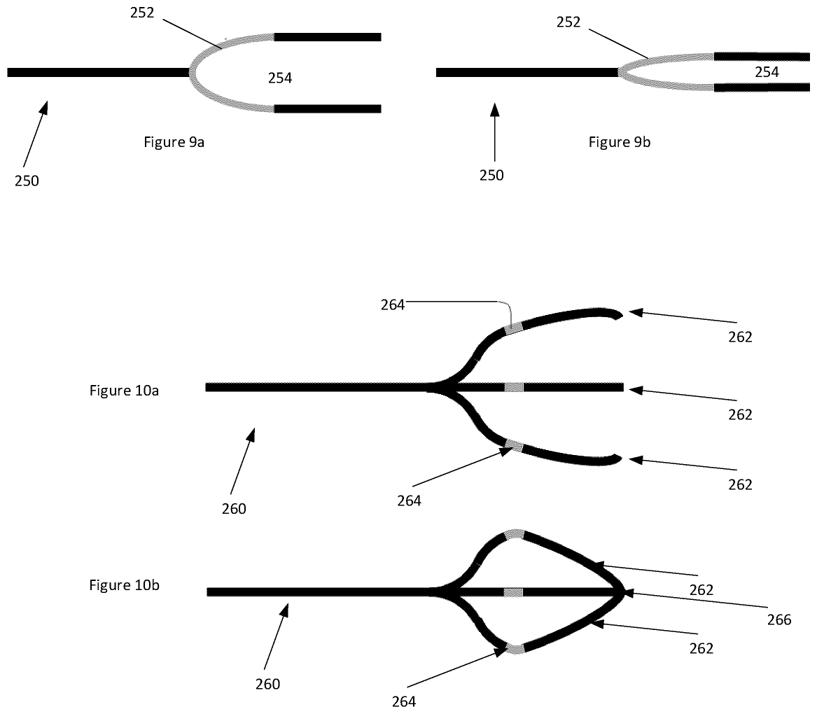

Another medical device of the disclosure that can be made from an SMA that is MMM processed is a heart stabilizer and/or clamp that may be used for occluding vessels. As shown in FIGS. 9a and 9b, the heart stabilizer or clamp 250 includes a set of processed regions 252 that are MMM processed. FIG. 9a is a schematic diagram of the clamp in an open position and FIG. 9b is schematic diagram of the clamp in a closed position. In the current embodiment, the processed regions 252 are near a base of a clamping area 254 such that the clamping area can open or close depending on a temperature applied to the clamp 250. In one embodiment, when the predetermined temperature is applied to the clamp, in response to the applied predetermined temperature, the processed regions actuate and move the clamp from the open position to the closed positon. In some embodiments, the processed regions 252 may have the same transformation, or responds to the same predetermined, temperatures. In other embodiments, the processed regions 252 may be made up of multiple smaller processed regions with differing transformation temperatures to create a “stepped” closing feature.

In some embodiments, the clamping areas may be measured and used as feedback to a control loop to control a position of the clamps when implanted or placed in a patient's body. By measuring the resistivity of the SMA, the amount of strain and the stage of the phase transformation can be inferred (such as described in, for example, US Patent Publication No. 2019/026466, published Aug. 29, 2019 which is hereby incorporated by reference). The control loop can act to control the clamping force so that it is not excessive to cause tissue harm or injury to a patient.

In another embodiment of a medical device made from SMA that has been MMM processed, the medical device may be a frame for an embolic filter such as schematically shown in FIGS. 10a and 10b. FIG. 10a is a schematic diagram of the frame in an open, collecting position and FIG. 10b is a schematic diagram of the frame in a closed, disposal position.

In the current embodiment, the frame 260 is made from SMA that has been MMM processed and includes a set of at least three arms 262 whereby each arm 262 includes a processed region 264. Although only one is shown, each arm may have multiple processed regions. The frame 260 may be MMM processed such that the processed regions 264 react to a same applied predetermined temperature or may be processed such that the processed regions 264 react to different applied predetermined temperatures. Due to the MMM processing, the processed regions 264 may be in a first, or open, position, at a first temperature (FIG. 10a) and then in a second, or closed, position, at a second or higher temperature (FIG. 10b). In the second position, the arms may close to secure captured embolic debris within the embolic filter.

In the open position, the arms may be expanded to conform to a vessel wall and, in the closed position, the distal ends of the arms may be positioned (via the reaction of the processed regions to a predetermined temperature), to come to a point 266 to secure the captured materials. In an alternative design, the entire frame and/or filter may be made up of SMA material with MMM processed regions within a band around the medical device, to open and close it as necessary dependent on an applied temperature.

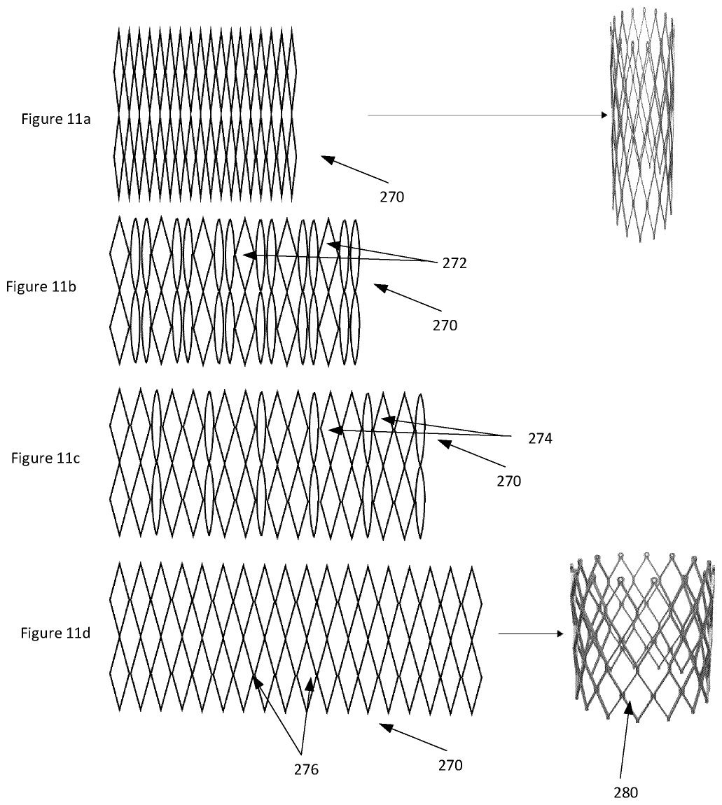

Turning to FIGS. 11a to 11d, further embodiments of a medical device manufactured from a SMA which is then MMM processed is shown. FIGS. 11a to 11d are directed at a stent which may be seen as a self-expanding stent that has been MMM processed, such as via a laser, to allow for expansion as a temperature applied to the stent is increased. In other embodiments, MMM processing may include the addition of an alloy to the medical device. FIG. 11a shows a stent in an initial deformable state; FIG. 11b shows the stent in a partially expanded state (in response to a slightly higher temperature compared to the temperature applied in FIG. 11a), FIG. 11c shows the stent in a more expanded state (in response to an applied temperature that is higher than the temperature applied in FIG. 11b; and FIG. 11d shows the stent in a fully expanded state (in response to an applied temperature that is higher than the temperature applied in FIG. 11c. A presence of multiple processed regions that react to different applied temperatures, allow for different areas of the stent to expand in stages to facilitate staged deployment of the stent within a patient's body. For example, as shown in FIGS. 11a to 11d, a stent 270 may be MMM processed to allow for three of stages of deployment whereby every third set of struts within the stent are processed with the same set of conditions. As will be understood, the number of stages of deployment may be determined by the manufacturer and can be any number greater than two.

Implementing a different number of stages of deployment is controlled by the MMM processing, such as will be described. In other embodiments, expansion of the struts may be in response to a lowering temperature instead of an increasing temperature or in response to specific operational conditions.

In the current embodiment, as shown, at the initial temperature, the stent is completely collapsed (FIG. 11a). At a slightly higher temperature than the temperature applied in FIG. 11a, such as a first predetermined temperature, every third cell 272 expands (FIG. 11b). At a further temperature (higher than the temperature applied in FIG. 11b) or a second predetermined temperature, a next set of struts 274 or cells adjacent the first set of cells expand (FIG. 11c). At yet another further temperature (higher than the temperature applied in FIG. 11c) or a third predetermined temperature, a further set of struts or cells 276 adjacent the second set of cells 274 expand (FIG. 11d) such that the stent can be seen as fully expanded 278.

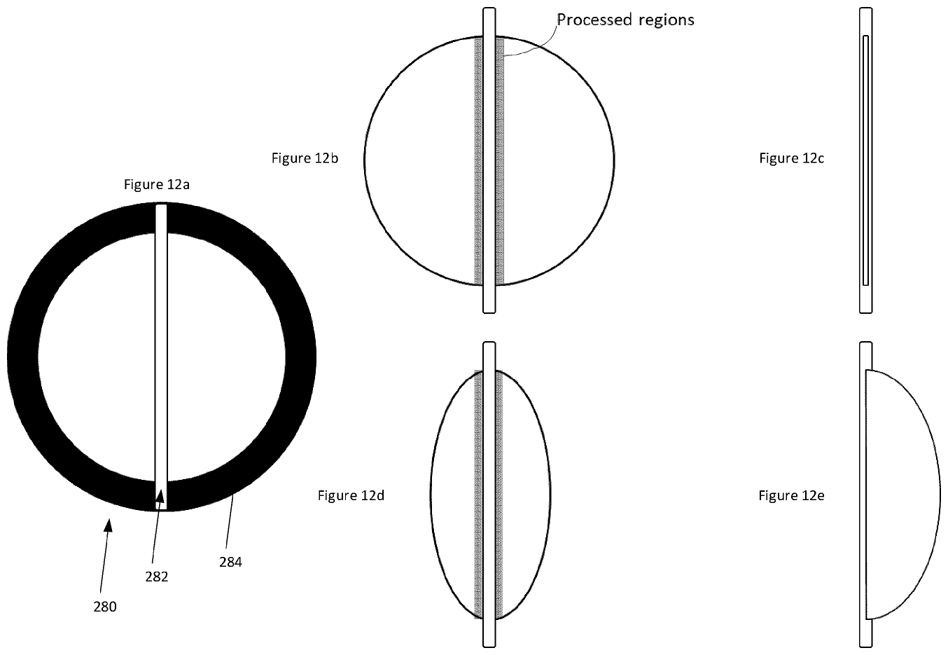

Turning to FIGS. 12a to 12e, another embodiment of a medical device manufactured from a SMA which is then MMM processed is shown. FIGS. 12a to 12e show a percutaneous valve. FIG. 12a is a schematic diagram of a butterfly valve, such as one made from NiTi, with a securement ring. FIG. 12b is a front view of the valve of FIG. 12a in a closed position, FIG. 12c is a side view of the valve of FIG. 12a in the closed position, FIG. 12d is a front view of the valve of FIG. 12a in an opened position and FIG. 12e is a side view of the valve in the opened position.

In one embodiment, the valve 280 is a thin sheet valve with a thicker central post 282 relative to the valve body 284 that secures the valve 280 to a securement ring 286 that may be attached to tissue, i.e., attached to the annulus of a resected heart valve. In one embodiment, the valve 280 and the post 282 may be made from a single sheet or may be made of a separate SMA sheet and wire. In alternative embodiments, the valve may be a bileaflet, monoleaflet, umbrella, duckbill, or other type of valve. In some embodiments, the valve may be MMM processed at locations that experience repeated strain to reduce localized stresses and improve the cycle life of the material.

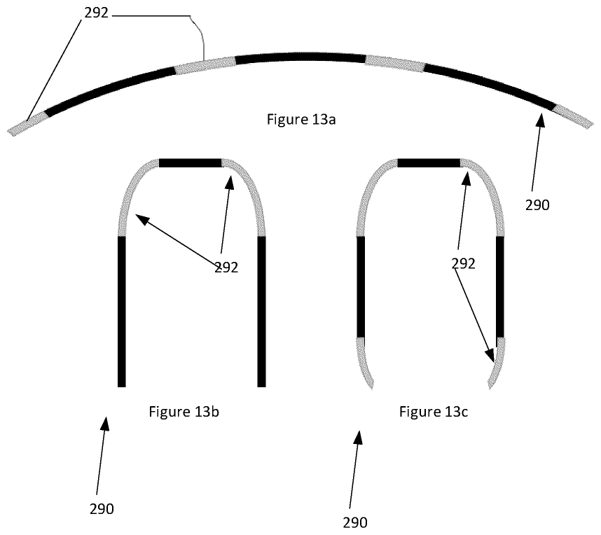

Turning to FIGS. 13a to 13c, another embodiment of a medical device manufactured from a SMA which is then MMM processed is shown. FIGS. 13a to 13c show a clip. The clip may be used to secure tissues or structures together and can be locally processed in regions that can bend and return to their remembered shape once heated. FIG. 13a shows the clip in an initial position, FIG. 13b shows the clip in a first memory position in response to a temperature higher than the temperature applied to the clip in FIG. 13a, and FIG. 13c shows the clip in a second memory position in response to a temperature higher than the temperature applied to the clip in FIG. 13b.

The clip 290, made from a SMA, includes a set of processed regions 292 that are a result of the SMA being passed through a MMM process. In use, in response to the application of at least one predetermined temperature, the processed regions provide a functionality or curve such as shown in FIGS. 13b and 13c. In some embodiments, the clip may be processed in one or several regions, with the same or different processing conditions for each to create varying responses to different applied temperatures. In some embodiments, as the clip is heated, it will return to its remembered shapes and maintain a constant force on the structures in which it is securing.

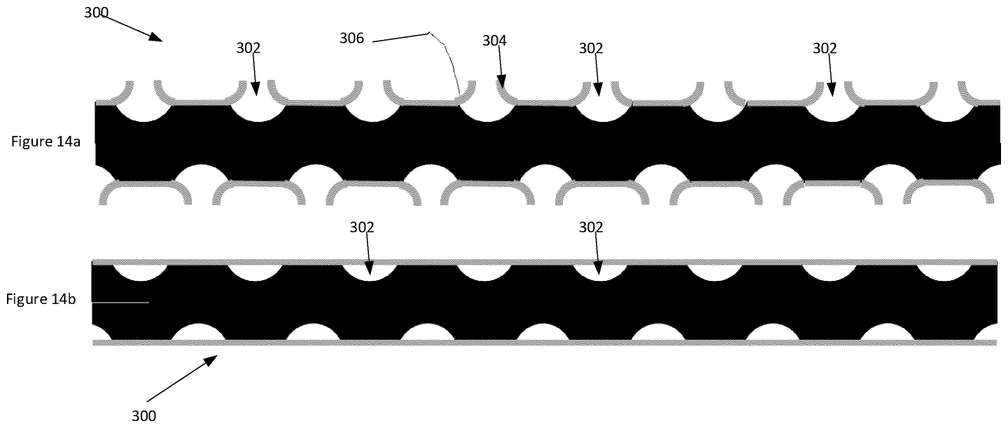

In another embodiment of the disclosure, the medical device may be a drug delivery device such as schematically shown in FIGS. 14a and 14b. The drug delivery device may be integrated or incorporated with other medical devices such as, but not limited to the devices disclosed above. Use of a MMM processed SMA in a drug delivery device may facilitate the local releasing of a drug or other substance or may facilitate the removal (via suction) of material from a patient's body. In some embodiments, the drug delivery device may relate to micro-pores that may be used as sites for cells which may attach or fuse to bone. FIG. 14a shows a drug delivery device (in the form of a wire) with micro-pores covered with valves in an open position and FIG. 14b shows the drug delivery device with the valves in a closed position.

In one embodiment, the drug delivery device 300 is made from a SMA material (such as a wire, sheet or tube of a SMA) with multiple micro-pores 302 and then covered with a sleeve 304 made from SMA that incorporates valves (by MMM processing the sleeve) in processed regions 306 to either contain and/or release a substance from within the micro-pores 302. The micro-pores 302 may be created using methods such as, but not limited to laser or EDM. In one embodiment, the micro-pores may be filled under pressure or vacuum conditions. When the micro-pores are filled under pressure, this substance or drug may be expelled when valves are opened. When the micro-pores are filled under a vacuum condition, opening of the valves results in a suction of cells, or other matter, into the micro-pores.

The sleeve 304 may be a separate component or created from the same SMA material as the device. The sleeve may be MMM processed such that at one temperature, the valves are opened, and at another temperature, the valves are closed with the open temperature being higher or lower than the closed temperature. In an alternative, the valves may be processed differently such that the transformation temperatures for each of the valves vary, allowing some valves to be opened while others are closed. In yet another embodiment, the valves may be MMM processed such that they may open varying amounts depending on the temperature, modulating the release of substance from each individual micro-pore.

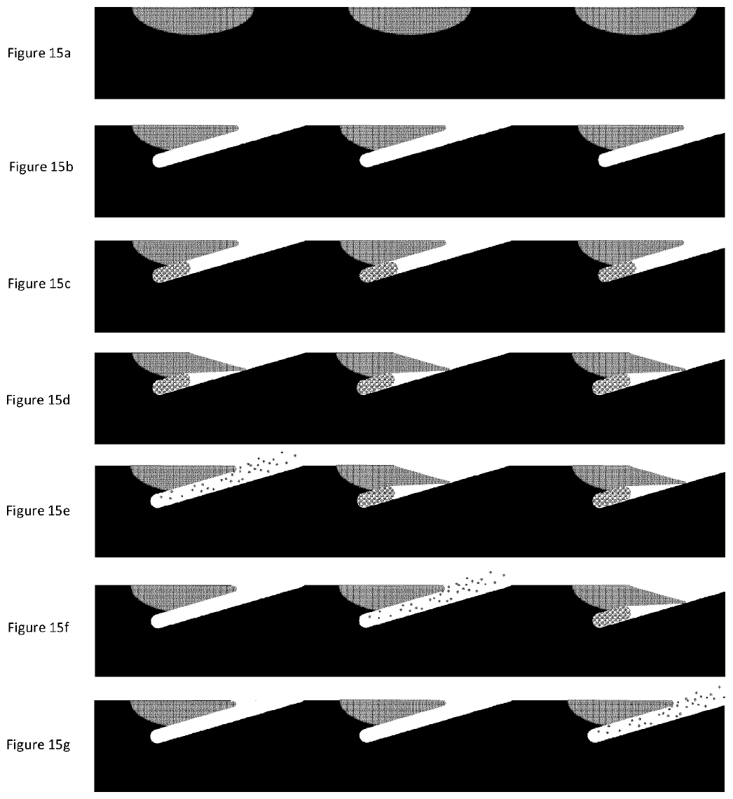

Another embodiment of a drug delivery device in accordance with the disclosure is schematically shown in FIGS. 15a to 15g. The drug delivery device of FIG. 15 may be seen as one with drug eluting functionality. FIG. 15a is a cross-section of the drug delivery device where the SMA has been MMM processed in various regions. FIG. 15b is a cross-section showing channels (which may function as valves) that are cut in the SMA material. FIG. 15c is a cross-section showing a drug or other substance being added via the channel. FIG. 15d is a cross-section showing the valves being deformed to a closed position. FIG. 15e is a cross-section showing the SMA being heated and the drug or substance being released from some of the valves. FIG. 15f is a cross-section showing the SMA being further heated such that the drug or substance is released from additional valves. FIG. 15g is a cross-section showing the SMA being further heated with the drug or substance being released from the remainder of the valves. As such, the drug delivery device of FIG. 15 includes processed regions that that have been MMM processed to respond or react to three different temperature levels. It is understood that the device may be designed to respond or reach to more or less than three different temperature levels.

The drug delivery device of FIG. 15 may be seen as an SMA with multiple micro-pores, located in regions processed using MMM processing or MMM laser processing, that may be shaped to cover the pores after deposition of a drug or other substance within the micro-pores. In some embodiments, the SMA can first be processed in various regions, either with the same processing conditions or varying processing conditions, such that the MMM or laser processed areas have a higher transformation temperature relative to the base material. After processing, micro pores may be cut at the processed regions, creating a flap-like valve of laser processed material (such as shown in FIG. 15b). A substance (or vacuum) may be added to the micro-pores and the valve may be closed while in their martensitic state. Upon heating above the austenitic transformation temperatures, the valves may open and allow release of the substance. If the valves have varying processing conditions, the amount of substance released will be dependent on the temperature that the SMA is heated to, allowing for a staged or timed release. In an alternative, the valves may be processed such that they may open varying amounts depending on the temperature, modulating the release of substance from each individual micro-pore.

In the preceding description, for purposes of explanation, numerous details are set forth in order to provide a thorough understanding of the embodiments. However, it will be apparent to one skilled in the art that these specific details may not be required. In other instances, well-known structures may be shown in block diagram form in order not to obscure the understanding. For example, specific details are not provided as to whether aspects of the embodiments described herein are implemented as a software routine, hardware circuit, firmware, or a combination thereof.

The above-described embodiments are intended to be examples only. Alterations, modifications and variations can be effected to the particular embodiments by those of skill in the art without departing from the scope, which is defined solely by the claims appended hereto.

We claim:

1. A surgical device comprising:
at least one processed region having altered properties to provide a predetermined functionality;
wherein the surgical device is made from a shape memory alloy (SMA); and
wherein the SMA is processed via a multiple memory material process to impart the at least one processed region into the SMA.

2. The surgical device of claim 1 further comprising at least two processed regions.

3. The surgical device of claim 2 wherein one of the at least two processed regions provides a functionality at a first predetermined temperature and another of the at least two processed regions provides a functionality at a second predetermined temperature.

4. The surgical device of claim 3 wherein the first predetermined temperature and the second predetermined temperature are different.

5. The surgical device of claim 3 wherein the first predetermined temperature and the second predetermined temperature are the same.

6. The surgical device of claim 1 wherein the surgical device is one of a guidewire, a catheter, an adjustable diameter ring, a tissue retractor, an annuloplasty band, a heart stabilizer, a clamp, a frame for an embolic filter, a stent, a valve, a clip or a drug delivery device.

7. The surgical device of claim 6 wherein when the surgical device is a catheter, the at least one processed region is near a tip of the catheter whereby when a predetermined temperature is applied to the catheter, the tip of the catheter bends at varying angles.

8. The surgical device of claim 6 wherein when the surgical device is a catheter, the at least one processed region includes pleats or folds whereby when a predetermined temperature is applied to the catheter, a diameter of the catheter increases to a target diameter.

9. The surgical device of claim 6 wherein when the surgical device is a guidewire, the at least one processed region includes at least two processed regions located on a diameter of the guidewire.

10. The surgical device of claim 9 wherein the at least two processed regions are opposite each other.

11. The surgical device of claim 6 wherein when the surgical device is an adjustable diameter ring, in response to a predetermined temperature, the at least one processed region bends to increase a diameter of the adjustable diameter ring.

12. The surgical device of claim 6 wherein when the surgical device is a clamp, in response to a predetermined temperature, the at least one processed region bends from an open position to a closed position.

13. The surgical device of claim 6 wherein when the surgical device is a frame for an embolic filter, in response to a predetermined temperature, the at least one processed region bends from an open position to a closed position.

14. The surgical device of claim 6 wherein when the surgical device is a stent, the at least one processed region includes multiple processed regions that respond to different predetermined temperatures.

15. The surgical device of claim 14 wherein when one of the different predetermined temperatures is applied to the stent, a portion of the multiple processed regions expands in response to the application of the different predetermined temperature.

16. The surgical device of claim 6 wherein when the surgical device is a drug delivery device that includes a set of micro-pores filled in a pressure condition, in response to a predetermined temperature, the at least one processed region exposes the micro-pores to release a drug stored in the set of micro-pores.

17. The surgical device of claim 6 wherein when the surgical device is a drug delivery device that includes a set of micro-pores filled under a vacuum condition, in response to a predetermined temperature, the at least one processed region exposes the micro-pores to create a suction to draw matter into the micro-pores.

18. The surgical device of claim 1 wherein the SMA is a form of a sheet, a wire or a tube.

19. The surgical device of claim 1 wherein the surgical device is post-processed after the SMA is processed via the multiple memory material process.

20. The surgical device of claim 19 wherein post processing comprises cold work, heat treatment, tumbling, thermal cycling, training or electropolishing.
